(19) United States
(12) Patent Application Publication
(10) Pub. No.: US 2022/0354833 A1
(43) Pub. Date: Nov. 10, 2022

(54) ALVELESTAT FOR USE IN THE TREATMENT OF GRAFT REJECTION, BRONCHIOLITIS OBLITERANS SYNDROME AND GRAFT VERSUS HOST DISEASE

(71) Applicants: MEREO BIOPHARMA 4 LIMITED, London (GB); DUKE UNIVERSITY, Durham, NC (US); NATIONAL CANCER INSTITUTE, Bethesda, MD (US)

(72) Inventors: Jacqueline PARKIN, London (GB); Anthony SUNG, Durham, NC (US); Steven Z. PAVLETIC, Bethesda, MD (US); Annie IM, Pittsburgh, PA (US); Noa G. HOLTZMAN, Bethesda, MD (US); Cody J. PEER, Bethesda, MD (US)

(73) Assignees: MEREO BIOPHARMA 4 LIMITED, London (GB); DUKE UNIVERSITY, Durham, NC (US); NATIONAL CANCER INSTITUTE, Bethesda, MD (US)

(21) Appl. No.: 17/753,706

(22) PCT Filed: Sep. 17, 2020
(86) PCT No.: PCT/EP2020/075948

§ 371 Date: Mar. 11, 2022

Related U.S. Application Data
(60) Provisional application No. 62/901,638, filed on Sep. 17, 2019.

(51) Int. Cl. A61K 31/444; A61P 37/06

(52) U.S. Cl. CPC A61K 31/444; A61P 37/06

(57) ABSTRACT

The invention relates to treatments for organ rejection, in particular to treatments for lung transplant associated bronchiolitis obliterans syndrome by administering a neutrophil elastase inhibitor, such as alvelestat. The invention also relates to treatments for graft versus host disease.

### CROSS REFERENCE TO RELATED APPLICATIONS

This application claims the benefit of priority of U.S. Provisional Patent Application Ser. No. 62/901638, filed 17 Sep. 2019. The contents of this application are incorporated herein by reference.

### GOVERNMENT LICENSE RIGHTS

This invention was made with government support under 1UG3TRO02448-01 awarded by the United States National Institutes of Health. The government has certain rights in the invention.

### FIELD OF THE INVENTION

The present invention relates to new methods for treating or preventing graft rejection and graft versus host disease comprising administering a neutrophil elastase inhibitor, in particular alvelestat or a pharmaceutically acceptable salt and/or solvate thereof, to a subject in need thereof.

### BACKGROUND TO THE INVENTION

Transplant of organs, bone marrow and human stem cells has advanced human health. However, transplantation is beset by the immune system's ability to recognise and react to non-self tissue. This is particularly a risk in allogeneic transplants when the tissue is from genetically similar, but not identical donors and there is a human leukocyte antigen (HLA) tissue type mismatch.

Graft rejection following solid organ transplantation can occur when the recipient's immune system (in particular the recipient's mature αβ T cells) recognises the foreign HLA antigens expressed on the donor organ's cells. It is dictated by host allo-responsiveness against mismatched donor antigens. Acute rejection typically occurs within the first weeks to several months after transplantation and is a major risk factor for the development of chronic rejection. Other risk factors for chronic rejection include infection. Chronic rejection typically develops within months to years after transplantation and is the major cause of long-term graft loss. Clinically, chronic rejection is characterized as a slow process resulting in the replacement of the allograft parenchyma with fibrous scar tissue.

Lung transplantation is an important treatment option for patients with advanced lung disease or irreversible pulmonary failure: around 3,500 lung transplantations are performed globally each year. However, acute lung rejection affects about a third of all lung transplant recipients within the first year after transplant, and may develop into chronic lung rejection (or chronic lung allograft dysfunction (CLAD)) which remains a major hurdle to long-term survival post lung transplantation. It is the leading cause of allograft loss and death for recipients of lung transplants surviving beyond 3 months post-transplant.

Lung Transplant associated Bronchiolitis Obliterans Syndrome (LT-BOS) is the most common form of CLAD, and manifests as a decline of lung function, which is often progressive. It is thought to be caused by inflammation, destruction and fibrosis of small airways in the lung allograft that leads to obliterative bronchiolitis (OB). Median survival after diagnosis is between 3-5 years [1].

Despite the high incidence, there is currently no adequate treatment for chronic graft rejection, in particular for LT-BOS. Current options for LT-BOS include immunosuppression therapy (often a triple combination), and neo-macrolides (such as azithromycin), as well as treatment of accompanying gastro-oesophageal reflux and infections. However, the evidence to support currently available therapies is limited, therapeutic response is typically poor, and the risk of serious adverse events is high: immunosuppression therapy greatly impairs immune reconstitution, which increases the risk of infections. As a last resort, lung re-transplantation may be considered, but outcomes are poor and donor organs are scarce. As a result, the ISHLT/ATS/ERS BOS Task Force concluded in 2014 that no currently available therapies have been proven to result in significant benefit in the prevention or treatment of LT-BOS [2].

As a further complication in transplant rejection, an immune response mounted against the recipient of an allograft by mature donor αβ T cells contained in the graft can lead to graft versus host disease (GVHD). Typically, GVHD is seen in the context of allogeneic haematopoietic stem-cell transplantation, although it can also occur in immunodeficient patients when they receive blood transfusions. Acute GVHD is characterized by damage to the skin, liver and the gastrointestinal tract, whereas chronic GVHD has more diverse manifestations and can resemble autoimmune syndromes. Standard of care is immunosuppression therapy, but as discussed above this carries a high risk of adverse events and increases the risk of infections [10].

There is therefore a need for new therapies for treating and preventing chronic graft rejection and GVHD, in particular LT-BOS.

### SUMMARY OF THE INVENTION

Surprisingly, inhibitors of neutrophil elastase (NE) such as alvelestat are useful in the treatment and prevention of GVHD and graft rejection, in particular in LT-BOS. This is unexpected because the main drivers of GVHD and graft rejection are typically considered to be T and B-lymphocytes rather than neutrophils. NE inhibitors had not previously been demonstrated to be effective against GVHD or graft rejection. In particular it had not previously been recognised that NE inhibitors could have utility in treating or preventing graft rejection in particular LT-BOS.

Thus the present invention provides a method for treating or preventing graft rejection, comprising administering an effective amount of a neutrophil elastase inhibitor, in particular alvelestat or a pharmaceutically acceptable salt and/or solvate thereof, to a subject in need thereof.

The present invention further provides a method for treating or preventing lung transplant associated bronchiolitis obliterans syndrome (LT-BOS), comprising administering an effective amount of a neutrophil elastase inhibitor, in particular alvelestat or a pharmaceutically acceptable salt and/or solvate thereof, to a subject in need thereof.

The present invention further provides a method for treating or preventing graft versus host disease (GVHD), comprising administering an effective amount of a neutrophil elastase inhibitor, in particular alvelestat or a pharmaceutically acceptable salt and/or solvate thereof, to a subject in need thereof.

The present invention also provides a method for treating or preventing bronchiolitis obliterans syndrome (BOS) associated with GVHD, e.g. BOS associated with haematopoietic stem cell transplant, comprising administering an effective amount of a neutrophil elastase inhibitor, in particular alvelestat or a pharmaceutically acceptable salt and/or solvate thereof, to a subject in need thereof.

### BRIEF DESCRIPTION OF THE FIGURES

FIG. 1 is a series of line graphs showing improved survival with alvelestat. BALB/c mice received 8.5 Gy TBI followed by transplantation from B10.D2 donors with either T-cell depleted bone marrow alone (TCDBM), TCDBM+2×10⁶ T cells (TCDBM+T2e6), or TCDBM+T2e6+one of three doses of alvelestat (20 mg/kg, 50 mg/kg, or 200 mg/kg) administered either in pre-mixed diet pellets (FIG. 1A, 1C, 1E) or as powder added to wet food (FIG. 1B, 1D, 1F). Endpoints include survival (FIG. 1A, 1B), body weight (FIG. 1C, 1D), and GVHD score (FIG. 1E, 1F). Individual experiments (n=5) were performed in duplicate and results combined in the figures (n=10/group).

FIG. 2 is a series of line graphs showing the effect of different doses of T cells on survival (FIG. 2A), bodyweight (FIG. 2B) and GVHD score (FIG. 2C). BALB/c mice received 8.5 Gy total body irradiation (TBI) followed by transplantation from B10.D2 donors with either T-cell depleted bone marrow alone (TCDBM), TCDBM+one of three different doses of T cells (1×10⁶ (TCDBM+T1e6), 1.5×10⁶ (TCDBM+T1.5e6), 2×10⁶ (TCDBM+T2e6)), or TCDBM+2×10⁶ T cells+alvelestat 20 mg/kg in pre-mixed diet pellets (TCDBM+T2e6+20 mg/kg diet) (n=5/group). Mice receiving TCDBM+T2e6+20 mg/kg had better survival than those receiving TCDB+T2e6 alone (p<0.001) and comparable survival to mice receiving lower doses of T cells.

FIG. 3 is a series of histological findings of GVHD in mice receiving T-cell depleted bone marrow+2×10⁶ T cells. Histological examination of skin from mice receiving TCDBM+T2e6 shows severe skin GVHD (A) with markedly hyalinized/fibrotic dermis, fat atrophy, loss of hair follicles and occasional epithelial apoptosis; likewise, histological examination of small intestinal mucosa from mice receiving TCDBM+T2e6 shows severe gut GVHD (B) with markedly reactive/regenerative epithelium with increased mitotic activity and apoptosis. In contrast, histological examination of skin from mice receiving TCDBM alone (no T cells) shows no sign of GVHD (C) with loose fibroconnective tissue within the dermis, ample subcutaneous adipose tissue and normal epithelium and hair follicles; likewise, histological examination of intestinal mucosa from mice receiving TCDBM alone shows no sign of GVHD (D) with appropriate cellularity and healthy appearing epithelium.

FIG. 4 is a plot showing the histological scoring of GI GVHD. Mice receiving drug (20 mg/kg) had markedly less GVHD as assessed by an experienced pathologist in a blinded fashion.

### DETAILED DESCRIPTION

The description below is made with the understanding that the present disclosure is to be considered as an exemplification of the claimed subject matter, and is not intended to limit the appended claims to the specific embodiments illustrated. The present disclosure provides reference to various embodiments and techniques. However, it should be understood that many variations and modifications may be made while remaining within the spirit and scope of the present disclosure. The headings used throughout this disclosure are provided for convenience and are not to be construed to limit the claims in any way. Embodiments illustrated under any heading may be combined with embodiments illustrated under any other heading.

Unless defined otherwise, all technical and scientific terms used herein have the same meaning as commonly understood by one of ordinary skill in the art. Throughout this specification and in the claims that follow, the following terms are defined with the following meanings, unless explicitly stated otherwise.

Unless the context requires otherwise, throughout the present specification and claims, the word “comprise” and variations thereof, such as, “comprises” and “comprising” are to be construed in an open, inclusive sense, i.e. as “including, but not limited to”.

Where the plural form is used for compounds, salts, and the like, this is taken to mean also a single compound, salt, or the like.

As used herein, the term “or” is generally employed in the sense as including “and/or” unless the context of the usage clearly indicates otherwise.

Also herein, the recitations of numerical ranges by endpoints include all numbers subsumed within that range (e.g., 1 to 5 includes 1, 1.5, 2, 2.75, 3, 3.80, 4, 5, etc.).

As used herein, the term “about” means the recited value ±10% of the recited value.

As used herein, “treatment” or “treating” is an approach for obtaining beneficial or desired results. For purposes of the present invention, beneficial or desired results include, but are not limited to, alleviation of a symptom and/or diminishment of the extent of a symptom associated with a disease or condition. “Treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject. The subject may be an individual at risk of developing the disease or disorder, such as an individual who has one or more risk factors known to be associated with development or onset of the disease or disorder. Thus, the term “preventing” in the present invention thus includes administering to a subject who will undergo transplantation, or has recently undergone transplantation without yet developing the associated condition.

As used herein, the term “therapeutically effective amount” or “effective amount” refers to an amount that is effective to elicit the desired biological or medical response, including the amount of a compound that, when administered to a subject for treating a disease, is sufficient to effect such treatment for the disease. The effective amount will vary depending on the particular compound, and characteristics of the subject to be treated, such as age, weight, etc. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

The term “solvate” is used herein to describe a molecular complex comprising the compound of the invention and a one or more pharmaceutically acceptable solvent molecules, for example, ethanol or water. The term “hydrate” is employed when the solvent is water and for the avoidance of any doubt, the term “hydrate” is encompassed by the term “solvate”.

The term “pharmaceutically acceptable salt” means a physiologically or toxicologically tolerable salt and includes, when appropriate, pharmaceutically acceptable base addition salts and pharmaceutically acceptable acid addition salts. For example, where a compound contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like. Hemisalts of acids and bases can also be formed, for example, hemisulfate and hemicalcium salts. For a review of suitable salts, see “Handbook of Pharmaceutical Salts: Properties, Selection and Use” by Stahl and Wermuth (Wiley-VCH, 2011).

“Pharmaceutically acceptable” or “physiologically acceptable” refer to compounds, salts, compositions, dosage forms etc. which are suitable for pharmaceutical use.

The term “subject” preferably refers to a human, normally who has received a transplant or is about to receive a transplant.

All documents referenced herein are each incorporated by reference in their entirety for all purposes.

### Alvelestat

The preferred neutrophil elastase inhibitor used in the invention is alvelestat.

Alvelestat is a potent, orally bioavailable neutrophil elastase inhibitor described in WO 2005/026123 A1 (Example 94, page 85) and [3], which are incorporated herein by reference in their entirety. Alvelestat has the chemical name N-{[5-(methanesulfonyl)pyridin-2-yl]methyl}-6-methyl-5-(1-methyl-1H-pyrazol-5-yl)-2-oxo-1-[3-(trifluoromethyl)phenyl]-1,2-dihydropyridine-3-carboxamide, and the following chemical structure:

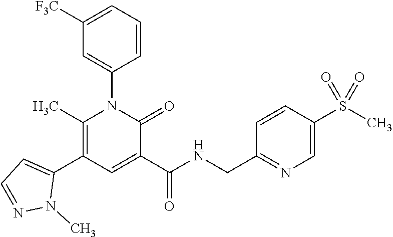 [SMILES: Cc1c(-c2ccnn2C)cc(C(=O)NCc2ccc(S(C)(=O)=O)cn2)c(=O)n1-c1cccc(C(F)(F)F)c1]

Alvelestat has also been referred to as AZD9668 and MPH996.

Alvelestat may be used in the invention in any pharmaceutically acceptable form, for example any free base form, salt form, and/or solvate form. Alvelestat or a pharmaceutically acceptable salt and/or solvate thereof may be present in any pharmaceutically acceptable physical form, suitably a solid form.

Certain salts of alvelestat are described in WO 2010/094964 A1, which is incorporated herein by reference in its entirety. Described salts of alvelestat include the tosylate, p-xylene-2-sulfonate, chloride, mesylate, esylate, 1,5-naphthalenedisulfonate and sulfate.

Preferably, alvelestat free base or alvelestat tosylate is used in the methods of the invention, more preferably alvelestat tosylate.

Alvelestat may also be used in any of the methods of the invention in a pharmaceutically acceptable prodrug form.

### Neutrophil Elastase Inhibitors

Neutrophil elastase (NE) is an enzyme that attacks and progressively damages lung tissue. Compounds that inhibit NE are reviewed in [13] and are known from various publications including WO2017207430, WO2017102674, WO2016050835, WO2016050835, WO2016016368, WO2016016366, WO2016016365, WO2016016364, WO2016016363, WO2015124563, WO2016020070, WO2015091281, WO2014135414, WO2014122160, WO2015096873, WO2015096872, WO2014029832, WO2014029831, WO2014029830, WO2014009425, WO2013084199, WO2013037809, WO2011103774, WO2011110858, WO2011110859, WO2011110852, WO2011039528, WO2010034996, WO2009061271, WO2009058076, WO2009060206, WO2007137080, WO2007137080, WO2007140117, WO2008036379, WO2008036379, WO9962538, WO9962538, WO9962514, WO9739028, WO9616080, WO9533763, WO9533762, WO9527055, WO9311760, WO9220357, WO9215605, WO9215605, WO03058237, WO03031574, WO03031574, WO2008030158, WO2007129963, WO2007129962, WO2006098684, WO2005026124, WO2005026123, WO2005021509, WO2005021512, WO2004043924, WO2009060158, WO2009037413, WO2009013444, WO2007129060, WO2007107706, WO2007107706, WO2006136857, WO2006082412, WO2006082412, WO9623812, WO9521855, WO9401455, WO9324519, WO9321214, WO9321210, WO9321213, WO9321209, WO9321212, WO2006070012, WO2005082863, WO2005082863, WO2005082864, WO9912933, WO9912933, WO9912931, WO9736903, WO2004020412, WO2008104752, WO2008097676, WO200809767, WO2008085608, each of which is incorporated by reference. Each of the neutrophil inhibitors described in these publications may be used in the methods of the invention, and is referred to as if it were individually disclosed herein for use in the methods of the invention.

In addition to the preferred neutrophil elastase inhibitor alvelestat, other exemplary neutrophil elastase inhibitors that may be used in the present invention include sivelestat, ONO-5046-Na, depelestat, Prolastin, KRP-109, DX-890, pre-elafin, MNEI, BAY 85-8501, POL6014, α1-AT, sirtinol, ONO-6818 (2-(5-amino-6-oxo-2-phenyl-1,6-dihydro-pyrimidin-1-yl)-N-[)R1R, 2R)-1-(5-tert-butyl-1,3,4-oxadiazol-2-yl)-1-hydroxy-3-methylbutan-2-yl]acetamide), elastatinal, SSR 69071 (2-[[6-methoxy-4-(1-methylethyl)-1,1-dioxo-3-oxo-1,2-benzisothiazol-2(3H)-yl]methoxy]-9-[2-(1-piperidinyl)ethoxy]-4H-pyrido[1,2-a]pyrimidin-4-one), and M0398 (N-(methoxysuccinyl)-L-alanyl-L-alanyl-L-prolyl-L-valine chrolomethylketone); and their pharmaceutically acceptable salts and/or solvates.

The term neutrophil elastase inhibitor includes all pharmaceutically acceptable forms of the compounds, for example all pharmaceutically acceptable salt, solvate, isomer, and prodrug forms.

In certain embodiments, the neutrophil elastase inhibitor is a small molecule compound, i.e. has a molecular weight of less than about 900 daltons.

Preferably the neutrophil elastase inhibitors are inhibitors of human neutrophil elastase.

Although many embodiments of this invention relate to alvelestat, it should be understood that for each and every embodiment described herein referring to “alvelestat”, the invention also provides a corresponding embodiment involving the use of “a neutrophil elastase inhibitor”.

### Treatments

The invention generally provides methods for treating or preventing graft rejection, acute graft rejection, chronic graft rejection, CLAD, LT-BOS, GVHD etc., in a subject in need thereof comprising administering to the subject an effective amount of a neutrophil elastase inhibitor, in particular alvelestat or a pharmaceutically acceptable salt and/or solvate thereof.

Accordingly, the present invention provides a method for treating or preventing graft rejection in a subject in need thereof comprising administering to the subject an effective amount of alvelestat or a pharmaceutically acceptable salt and/or solvate thereof.

Graft rejection may also be referred to as organ transplant rejection.

The methods described herein are useful for treating or preventing acute graft rejection. In certain embodiments, the method is for treating acute graft rejection. In other embodiments, the method is for preventing acute graft rejection.

The methods described herein are useful for treating or preventing chronic graft rejection. In certain embodiments, the method is for treating chronic graft rejection. In other embodiments, the method is for preventing chronic graft rejection.

The graft may comprise any solid organ, in particular those solid organs that are frequently transplanted. So, the graft may comprise one or more organs selected from the group consisting of kidney, heart, liver, lung and pancreas.

Chronic rejection of cardiac (i.e. heart) allografts is manifested by cardiac allograft vasculopathy (CAV). This is typically characterized by occlusion of coronary vessels. The 5-year incidence of CAV is 30-40%. Accordingly, the present invention provides a method for treating or preventing CAV in a subject in need thereof comprising administering to the subject an effective amount of alvelestat or a pharmaceutically acceptable salt and/or solvate thereof.

Chronic rejection of kidney allografts is manifested by cardiac allograft nephropathy (CAN). CAN is the leading cause of renal function deterioration and accounts for nearly 40% of the graft loss at 10 years. Accordingly, the present invention provides a method for treating or preventing CAN in a subject in need thereof comprising administering to the subject an effective amount of alvelestat or a pharmaceutically acceptable salt and/or solvate thereof.

In preferred embodiments, the graft comprises a lung. The graft may be a single or a double lung transplant. The graft may be a heart-lung transplant. Thus, the present invention provides a method for treating or preventing lung transplant rejection in a subject in need thereof comprising administering to the subject an effective amount of alvelestat or a pharmaceutically acceptable salt and/or solvate thereof. It may be acute lung transplant rejection or chronic lung transplant rejection.

Chronic rejection of lung allografts is manifested by chronic lung allograft dysfunction (CLAD). Accordingly, the present invention provides a method for treating or preventing CLAD in a subject in need thereof comprising administering to the subject an effective amount of alvelestat or a pharmaceutically acceptable salt and/or solvate thereof.

The most common phenotype of CLAD is lung transplant associated bronchiolitis obliterans syndrome (LT-BOS). Bronchiolitis obliterans may also be referred to as obliterative bronchiolitis. Typical characteristics include an obstructive pulmonary function defect and air trapping/mosaic attenuation on expiratory CT. Accordingly, the present invention provides a method for treating or preventing LT-BOS in a subject in need thereof comprising administering to the subject an effective amount of alvelestat or a pharmaceutically acceptable salt and/or solvate thereof.

Methods of preventing LT-BOS according to the invention are particularly useful for subjects at risk of LT-BOS. Such subjects may possess one or more risk factors selected from the group consisting of primary graft dysfunction, gastro-oesophageal reflux, infection, airway ischemia, acute rejection, lymphocytic bronchiolitis, infection and colonization with micro-organisms (e.g., Pseudomonas aeruginosa and Aspergillus fumigatus), donor and recipient genetics, particulate matter and presence of HLA antibodies, or antibodies to self-antigens (such as K-α1 tubulin and collagen V).

The present invention provides a method for treating or preventing GVHD in a subject in need thereof comprising administering to the subject an effective amount of alvelestat or a pharmaceutically acceptable salt and/or solvate thereof. GVHD manifests following tissue transplantation. In some embodiments, the transplant is selected from the group consisting of skin, hematopoietic stem cells, blood and bone marrow. In preferred embodiments, the transplant is hematopoietic stem cells.

The GVHD may be acute graft versus host disease (aGVHD). The disease may be chronic graft versus host disease (cGVHD). Acute GVHD is typically characterized by damage to the skin, liver and the gastrointestinal tract, whereas chronic GVHD typically has more diverse manifestations and can resemble autoimmune syndromes with, for example, eosinophilic fasciitis, scleroderma-like skin disease and salivary and lacrimal gland involvement.

An additional embodiment provides a method for inhibiting the onset of symptoms of GVHD, including aGVHD and cGVHD, the method comprising administering a pharmaceutically effective amount of alvelestat or a pharmaceutically acceptable salt and/or solvate thereof to a recipient of a transplantation of allogenic hematopoietic stem cells.

In the above methods relating to GVHD, the GVHD may be characterised by damage to one or more selected from the group consisting of the eyes, joints, fascia, genital organ, lung, liver, skin, or gastrointestinal tract (e.g. mouth, oesophagus).

In particular, in the above methods relating to GVHD, the GVHD may be characterised by damage to one or more selected from the group consisting of the lung, liver, skin, or gastrointestinal tract.

Chronic GVHD may be classified according to various criteria. The 2005 and 2014 National Institutes of Health Consensus Development Projects on Criteria for Clinical Trials in Chronic GVHD standardized the terminology around chronic GVHD classification systems [16].

One classification system is the NIH severity score, which is divided into mild, moderate or severe based on the number and severity of involved organs. Accordingly, in methods of the invention relating to treating cGVHD, the subject may have cGVHD which is mild, moderate or severe according to the NIH severity score. In particular, the cGVHD may be moderate or severe, typically severe. Furthermore, methods for improving the cGVHD severity score in a subject with cGVHD are provided herein, comprising administering alvelestat or a pharmaceutically acceptable salt and/or solvate thereof.

Another classification system based on patient-reported outcomes is the Lee cGVHD Symptom Scale [17]. Accordingly, methods for improving the Lee cGVHD Symptom Scale in a patient with cGVHD are provided herein, comprising administering alvelestat or a pharmaceutically acceptable salt and/or solvate thereof. In particular, methods for improving the Lee cGVHD Symptom Scale lung score in a subject with cGVHD affecting a lung are provided herein, comprising administering alvelestat or a pharmaceutically acceptable salt and/or solvate thereof.

The invention also provides a method for treating or preventing bronchiolitis obliterans syndrome (BOS) associated with GVHD, comprising administering an effective amount of alvelestat or a pharmaceutically acceptable salt and/or solvate thereof to a subject in need thereof. In preferred embodiments, the subjects are receiving hematopoietic stem cell transplantation. Also provided is alvelestat or a pharmaceutically acceptable salt thereof for use in treating or preventing bronchiolitis obliterans syndrome (BOS) associated with GVHD. Also provided is the use of alvelestat or a pharmaceutically acceptable salt thereof for the manufacture of a medicament for treating or preventing bronchiolitis obliterans syndrome (BOS) associated with GVHD.

In the described methods, the NE inhibitor, in particular alvelestat or a pharmaceutically acceptable salt and/or solvate thereof, may be administered to the subject prior to transplantation. For example, alvelestat administration may start 14 days, 7 days, 3 days, 2 days, or 1 day prior to transplantation.

In the described methods, the NE inhibitor, in particular alvelestat or a pharmaceutically acceptable salt and/or solvate thereof, may be administered to the subject after transplantation. For example, the alvelestat administration may start on the day of transplantation or 1 day, 2 days, 3 days, 7 days, or 14 days after transplantation.

Methods according to the invention relating to BOS, in particular LT-BOS, may also comprise improving one or more pulmonary function parameters in a subject.

In particular, methods according to the invention may improve the FEV1 of a subject. Forced expiratory volume (FEV) is the expiratory volume of air from a maximally forced effort measured over a set period of time, e.g. 1 second (FEV1).

In particular, methods according to the invention may improve the FEV1% predicted of a subject. FEV1% predicted is the ratio of FEV1 in a subject to the predicted FEV1 of a normal person of similarly matched race or ethnicity, gender, age, height and weight, expressed as a percentage.

Accordingly, also provided is a method for increasing FEV1% predicted in a subject with LT-BOS, by administering an effective amount of a NE inhibitor, in particular alvelestat or a pharmaceutically acceptable salt and/or solvate thereof.

In particular embodiments, treatment with an effective amount of alvelestat or a pharmaceutically acceptable salt and/or solvate thereof increases FEV1% predicted by at least about 1%, 1.5%, 2.0%, 2.5%, 3.0%, 4.0%, 5.0%, 6.0%, 7.0%, 8.0%, 9.0%, 10%, 15%, 20%, 30%, 40% or 50% A compared to a baseline FVC % predicted measurement. In further embodiments, treatment with an effective amount of alvelestat or a pharmaceutically acceptable salt and/or solvate thereof prevents FEV1% from worsening.

Methods according to the invention relating to LT-BOS may also comprise improving the BOS grade of a subject. The BOS classification scheme, adopted in 1993, provided a staging system based on the severity of lung function decline after transplant and has been used for clinical decision-making and research purposes. This staging system was most recently modified in 2002 [2]. The BOS classification scheme from 2002 is used according to the invention:

Grading (staging) of bronchiolitis obliterans syndrome (BOS)^{#}

| BOS grade | Spirometry % of baseline^{¶} |
|---|---|
| 0 | FEV₁ > 90% and FEF₂₅₋₇₅ % > 75% |
| 0-p⁺ | FEV₁ 81-90% and/or FEF_{25-75%} ≤ 75% |
| 1 | FEV₁ 66-80% |
| 2 | FEV₁ 51-65% |
| 3 | FEV₁ ≤ 50% |

FEV1: forced expiratory volume in 1 s; FEF_{25-75%}: forced expiratory flow at 25-75% of forced vital capacity.
^{#}Other causes of lung function decline must be excluded (e.g. acute rejection, infection, native lung problems for single lung recipients, excessive recipient weight gain, anastomotic dysfunction, respiratory muscle dysfunction, effusion, or technical problems such as erroneous measurements due to device dysfunction);
^{¶}baseline is defined as the average of the two best FEV₁ (or FEF_{25-75%}) values (≥3 weeks apart) following functional recovery and stabilisation post-lung transplantation;
⁺in Grade (stage) 0-p the “p” denotes “probable” early BOS and is used to indicate 10-20% decline in baseline FEV1 that is likely due to an early stage of BOS that does not meet criteria for BOS Grade 1.

Thus in embodiments relating to treatment of LT-BOS, treatment with an effective amount of alvelestat or a pharmaceutically acceptable salt and/or solvate thereof improves the BOS grading by at least 1 grade. In further embodiments relating to treatment of LT-BOS, treatment with an effective amount of alvelestat or a pharmaceutically acceptable salt and/or solvate thereof prevents the BOS grading from worsening.

Diagnosis of BOS can be carried out by the skilled clinician. Imaging tests, such as high resolution chest CT scan, and pulmonary function tests can help detect BOS. Chest x-rays may also be used. A surgical lung biopsy can also be carried out to diagnose the BOS. Lung biopsies may show small airway involvement with fibrinous obliteration of the lumen. Bronchoalveolar lavage (BAL) may show neutrophilic and/or lymphocytic inflammation.

Also provided in this invention are methods for treating or preventing BOS associated with connective tissue disease, systemic lupus erythematosus, rheumatoid arthritis, infection, toxic fume exposure, or Stevens-Johnson syndrome, comprising administering an effective amount of a neutrophil elastase inhibitor, in particular alvelestat or a pharmaceutically acceptable salt and/or solvate thereof, to a subject in need thereof.

The patient to be treated in the methods of the present invention may in some embodiments have a baseline FEV1 of 30% of predicted FEV1 or higher, e.g. a baseline FEV1 of 35% or higher, or 40% or higher. The patient may have a baseline FEV1 of 20-90% of predicted FEV1, for example of 30-80%, 35-75%, or 40-50%.

Without wishing to be bound by theory, it is considered that alvelestat is beneficial in the methods of the invention due to its ability to inhibit neutrophil elastase. Accordingly, the invention also provides methods of inhibiting neutrophil elastase in a subject suffering from, or at risk of, any of the conditions described herein, including graft rejection or GVHD, in particular LT-BOS, comprising administering an effective amount of a neutrophil elastase inhibitor, in particular alvelestat or a pharmaceutically acceptable salt and/or solvate thereof to the subject. Also provided are each of the above methods for treating or preventing any of the conditions described herein, including graft rejection or GVHD, in particular LT-BOS, by inhibiting neutrophil elastase, comprising administering an effective amount of a neutrophil elastase inhibitor, in particular alvelestat or a pharmaceutically acceptable salt and/or solvate thereof to the subject.

The present invention also relates to methods for improving lung function in a subject referred to in this disclosure, in particular a subject with GVHD affecting the lungs, such as chronic GVHD, said method comprising administering an effective amount of a NE inhibitor, in particular alvelestat or a pharmaceutically acceptable salt and/or solvate thereof to the subject.

The present invention also relates to methods for preventing worsening of lung function in a subject referred to in this disclosure, in particular a subject with GVHD affecting the lungs, such as chronic GVHD, said method comprising administering an effective amount of a NE inhibitor, in particular alvelestat or a pharmaceutically acceptable salt and/or solvate thereof to the subject.

The present invention also relates to methods for stabilising lung function in a subject referred to in this disclosure, in particular patients with GVHD affecting the lungs, such as chronic GVHD, said method comprising administering an effective amount of a NE inhibitor, in particular alvelestat or a pharmaceutically acceptable salt and/or solvate thereof to the subject.

The present invention also relates to methods for preventing progression or worsening of disease in a subject referred to in this disclosure, in particular a subject with GVHD, such as cGVHD. The present invention also relates to methods for stabilising disease in a subject referred to in this disclosure, in particular a subject with GVHD, such as cGVHD.

The present invention also relates to methods for preventing progression of disease in a subject referred to in this disclosure, in particular a subject with GVHD, such as cGVHD. The present invention also relates to methods for stabilising disease in a subject referred to in this disclosure, in particular a subject with GVHD, such as cGVHD.

Also provided is alvelestat or a pharmaceutically acceptable salt and/or solvate thereof for use in treating or preventing graft rejection, for example chronic or acute graft rejection. Also provided is alvelestat or a pharmaceutically acceptable salt thereof for use in treating or preventing lung transplant associated bronchiolitis obliterans syndrome. Also provided is alvelestat or a pharmaceutically acceptable salt thereof for use in treating or preventing GVHD.

Also provided is the use of alvelestat or a pharmaceutically acceptable salt and/or solvate thereof for the manufacture of a medicament for treating or preventing graft rejection, for example chronic or acute graft rejection. Also provided is the use of alvelestat or a pharmaceutically acceptable salt thereof for the manufacture of a medicament for treating or preventing lung transplant associated bronchiolitis obliterans syndrome. Also provided is the use of alvelestat or a pharmaceutically acceptable salt thereof for the manufacture of a medicament for treating or preventing GVHD.

### Dosing

For the above mentioned therapeutic indications, the dose of the neutrophil inhibitor to be administered, in particular alvelestat or a pharmaceutically acceptable salt and/or solvate thereof, will depend on the disease being treated, the severity of the disease, the mode of administration, the age, weight and sex of the patient. Such factors may be determined by the attending physician. However, in general, satisfactory results are obtained when the compounds are administered to a human at a daily dosage of between 0.1 mg/kg to 100 mg/kg (measured as the active ingredient).

Suitably the daily dose is from 0.5 to 1000 mg per day, for example from 50 to 800 mg per day, in particular 50 to 600 mg per day, more particularly 120 mg to 550 mg, even more particularly 200 to 500 mg. For example the daily dose is about 240, 270, 300, 330, 360, 390, 420, 450 or 480 mg per day. The dose may be administered as a single dose or as a divided dose, for example wherein the total daily dose is divided in to two or more fractions, administered during the day. A dose may be administered daily, or multiple times a day (for example twice daily), or multiple times a week, or monthly, or multiple times a month.

In a particular embodiment alvelestat or a pharmaceutically acceptable salt and/or solvate thereof is administered twice a day (BID dosing). In a further embodiment alvelestat or a pharmaceutically acceptable salt and/or solvate thereof is administered twice a day, wherein each dose is equivalent to up to 240 mg of alvelestat free base, for example 60 mg twice a day, 90 mg twice a day, 120 mg twice a day, 150 mg twice a day, 180 mg twice a day, 210 mg twice a day, or 240 mg twice a day. In particular, 120 mg is administered twice a day or 240 mg is administered twice a day.

Compounds may be administered to an individual in accordance with an effective dosing regimen for a desired period of time or duration, such as at least one week, at least about one month, at least about 2 months, at least about 3 months, at least about 6 months, at least about 12 months, at least about 24 months, or longer. For example, the compound may be administered on a daily or intermittent schedule for the duration of the subject's life.

The dose of alvelestat or a pharmaceutically acceptable salt and/or solvate thereof may be administered according to a dosage escalation regime in all methods of the invention. This allows safe titration up to the standard daily dose of alvelestat, e.g. of 240 mg twice daily. For example, a dosage escalation regime up to a standard 240 mg twice daily dose of alvelestat according to the invention comprises administration of alvelestat or a pharmaceutically acceptable salt and/or solvate thereof at a dose of 60 mg of alvelestat twice daily for a first period of time, followed by 120 mg twice daily for a second period of time, followed by 180 mg twice daily for a third period of time, and 240 mg twice daily thereafter. The first, second and third periods may each be from 10-20 days, e.g. each about two weeks. In particular, alvelestat or a pharmaceutically acceptable salt and/or solvate thereof is administered at 60 mg twice daily for two weeks, followed by 120 mg twice daily for two weeks, followed by 180 mg twice daily for two weeks, and 240 mg twice daily thereafter. Doses are referred to as the equivalent amount of alvelestat free base.

### Compositions

The neutrophil inhibitor, in particular alvelestat or a pharmaceutically acceptable salt and/or solvate thereof, is administered to a subject in the form of a pharmaceutical composition.

Accordingly, the invention provides a method of treating or preventing any of the conditions described herein comprising administering a pharmaceutical composition comprising an effective amount of a neutrophil inhibitor, in particular alvelestat or a pharmaceutically acceptable salt and/or solvate thereof, and one or more pharmaceutically acceptable excipients, to a subject in need thereof.

Pharmaceutical compositions may be prepared with one or more pharmaceutically acceptable excipients which may be selected in accord with ordinary practice.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans. All compositions may optionally contain excipients such as those set forth in the Shesky et al, Handbook of Pharmaceutical Excipients, 8^{th} edition, 2017. Excipients can include ascorbic acid and other antioxidants, chelating agents such as EDTA, carbohydrates such as dextrin, hydroxyalkylcellulose, hydroxyalkylmethylcellulose, stearic acid and the like.

Pharmaceutical compositions include those suitable for various administration routes, including oral administration. The compositions may be presented in unit dosage form and may be prepared by any of the methods well known in the art of pharmacy. Such methods include the step of bringing into association the active ingredient (e.g., a compound of the present disclosure or a pharmaceutical salt thereof) with one or more pharmaceutically acceptable excipients. The compositions may be prepared by uniformly and intimately bringing into association the active ingredient with liquid excipients or finely divided solid excipients or both, and then, if necessary, shaping the product. Techniques and formulations generally are found in Remington: The Science and Practice of Pharmacy, 22^{nd} Edition, 2012.

A preferred pharmaceutical composition is a solid dosage form, including a solid oral dosage form, such as a tablet. Tablets may contain excipients including glidants, fillers, binders and the like.

In effecting the methods described herein, the pharmaceutical compositions can be administered in any form and route which makes the compound bioavailable. Thus, the pharmaceutical compositions can be administered by a variety of routes, including oral and parenteral routes, more particularly by inhalation, subcutaneously, intramuscularly, intravenously, transdermally, intranasally, rectally, vaginally, occularly, topically, sublingually, and buccally, intraperitoneally, intravenously, intraarterially, transdermally, sublingually, intramuscularly, rectally, transbuccally, intranasally, intraadiposally, intrathecally and via local delivery for example by catheter or stent. Preferably, the pharmaceutical compositions are administered orally.

When used for oral use, tablets, troches, lozenges, aqueous or oil suspensions, dispersible powders or granules, emulsions, hard or soft capsules, syrups or elixirs may be prepared. Compositions described herein that are suitable for oral administration may be presented as discrete units (a unit dosage form) including but not limited to capsules, cachets or tablets each containing a predetermined amount of the active ingredient. Preferably, the pharmaceutical composition is a tablet.

Aqueous compositions may be prepared in sterile form, and when intended for delivery by other than oral administration generally may be isotonic.

The amount of active ingredient that may be combined with the inactive ingredients to produce a dosage form may vary depending upon the intended treatment subject and the particular mode of administration.

### Combination Therapy

In the present invention, the methods may further include the step of administering to the subject one or more additional therapeutic agents. The administration of the one or more additional therapeutic agents may occur prior to, concurrently with, or after the administration of the neutrophil inhibitor.

Additional therapeutic agents include immunosuppressive agents, anti-infective agents, anti-inflammatory agents, and pain relievers.

In particular embodiments, the one or more additional therapeutic agent are immunosuppressive agents. For example, one, two, or preferably three immunosuppressive agents may be administered.

The immunosuppressive agents may, for example, be selected from the group consisting of corticosteroids (e.g. methylprednisolone, prednisone, prednisolone, budesonide, dexamethasone), janus kinase inhibitors (e.g. tofacitinib), calcineurin inhibitors (e.g. cyclosporine, tacrolimus), mTOR inhibitors (e.g. sirolimus, everolimus, temsirolimus), biologics (e.g. abatacept, adalimumab, anakinra, certolizumab, etanercept, golimumab, infliximab, ixekizumab, natalizumab, rituximab, secukinumab, tocilizumab, ustekinumab, vedolizumab), monoclonal antibodies (e.g. basiliximab, daclizumab), tyrosine kinase inhibitors (e.g. imatinib), thalidomide, pentostatin, azathioprine, mycophenolate and methotrexate.

In certain embodiments, the methods further include the step of administering to the subject a triple combination of immunosuppressive agents, for example tacrolimus, mycophenolate and a corticosteroid.

The one or more additional therapeutic agents may be anti-infective agents. Anti-infective agents include antibiotics, antifungals, anthelmintics, antimalarials, antiprotozoals, antituberculosis agents, and antivirals.

The one or more additional therapeutic agents may be selected from the group of prednisone, methylprednisone, budesonide, beclomethasone dipropionate, cyclosporine, tacrolimus, sirolimus, mycophenolate mofetil, tilomisole, imuthiol, antithymocyte globulin, azathioprine, azodiacarbonide, bisindolyl maleimide VIII, brequinar, chlorambucil, CTLA4-Ig, cyclophosphamide, deoxyspergualin, dexamethasone, leflunomide, mercaptopurine, 6-mercaptopurine, methotrexate, methylprednisolone, mizoribine, mizoribine monophosphate, muromonab CD3, mycophenolate mofetil, OKT3, rho (D) immune globin, vitamin D analogs, MC1288), daclizumab, infliximab, rituximab, tocilizumab alemtuzumab, methotrexate, antithymocyte globulin, denileukin diftitox, Campath-1H, keratinocyte growth factor, abatacept, remestemcel-L suberoylanilide hydroxamic acid, pentostatin, thalidomide, imatinib mesylate, cyclophosphamide, fludarabine, OKT3, melphalan, thiopeta, and lymphocyte immune globulin, anti-thymocyte, and globulin.

It is also understood that each of the agents administered individually or combined in a combination therapy or regimen may be administered at an initial dose that may then over time be reduced by a medical professional to reach a lower effective dose. For instance, in the combinations and regimens herein, systemic glucocorticosteroids (corticosteroids), such as prednisone and methyl prednisone may be administered to a human patient at a dose of from about 1-2 mg/kg/day. Initial daily doses for mTOR agents include sirolimus at 2-40 mg given once daily and everolimus at 0.25-1 mg given twice daily. Initial daily doses for calcineurin agents include tacrolimus at from about 0.025-0.2 mg/kg/day and cyclosporine at from about 2.5-9 mg/kg/day. Mycophenolate mofetil (CellCept®) may be administered at an initial daily dose of about 250-3,000 mg/day. Each of these agents may be administered in combination with a pharmaceutically effective amount of a Syk inhibitor as described herein following hematopoietic cell transplant. In different embodiments herein, agents useful in treating GVHD may be administered topically to a human in need of such treatment, such as in the form of a topical ointment or cream or in an eye drop formulation.

The present invention also provides methods for treating GVHD further including the step of administering light therapy (also known as extracorporeal photopheresis).

### EXAMPLES

Embodiments provided herein may be more fully understood by reference to the following examples. These examples are meant to be illustrative of methods provided herein, but are not in any way limiting. It will be apparent to those skilled in the art that various changes and modifications may be made. Such modifications are also intended to fall within the scope of the appended claims.

Alvelestat used in the following examples may be synthesised according to WO 2005/026123 A1 (Example 94, page 85).

### Example 1—Alvelestat is a Potent and Specific Inhibitor of Neutrophil Elastase (NE)

The following results were obtained as discussed further in [3].

Alvelestat has a high binding affinity for human NE (K_{D}=9.5 nM) and potently inhibits NE activity. The calculated ₚIC₅₀ (IC₅₀) and Kᵢ values for alvelestat for human NE are 7.9 (12 nM) and 9.4 nM, respectively.

Alvelestat is at least 600-fold more selective for human NE compared with another serine protease cathepsin G, and at least 1900-fold more selective for human NE comprised with other serine proteases (proteinase-3, chymotrypsin, pancreatic elastase and trypsin).

Alvelestat shows good crossover potency for NE from other species, including mice.

The ₚIC₅₀ (IC₅₀) values in whole-blood, cell-associated, and explosive-release assays were 7.36 (44 nM), 7.32 (48 nM), and 7.30 (50 nM), respectively.

The results of the studies presented show that alvelestat is a specific, potent, and rapidly reversible inhibitor of human NE. The potent inhibitory activity of alvelestat on NE in biochemical assays was confirmed in whole-blood and cell-based systems.

### Example 2—Alvelestat Shows a Protective Effect Against GVHD

This preclinical study was conducted to evaluate the efficacy of alvelestat in the prevention of GVHD. Preclinical murine studies are reasonable given that alvelestat has similar potency for murine and human NE (pIC50 6.5 vs. 7.9) [3].

The murine model of GVHD used in this study is described in [4]. BALB/c recipients received lethal irradiation (8.5 Gy total body irradiation) followed by transplantation with T-cell depleted bone marrow +/− purified T cells from B10.D2 donors. Negative controls received T-cell depleted bone marrow (TCDBM) only (no T cells, no GVHD), while positive controls received TCDBM+2×10⁶ T cells (100% fatal GVHD). In the treatment arms, mice received alvelestat at 20, 50, and 200 mg/kg per day, days −1 to 45 through a pre-mixed custom diet or added as a powder to wet food. These doses are based on theoretical considerations and previous work in rodents (e.g. [5]). This schedule was chosen to ensure adequate drug levels before irradiation (Day 0) through the period of GVHD mortality. Survival (primary endpoint), body weight, and GVHD score (clinical and histological) ([4], [6]) were monitored. To ensure scientific rigor, experiments were performed in duplicate.

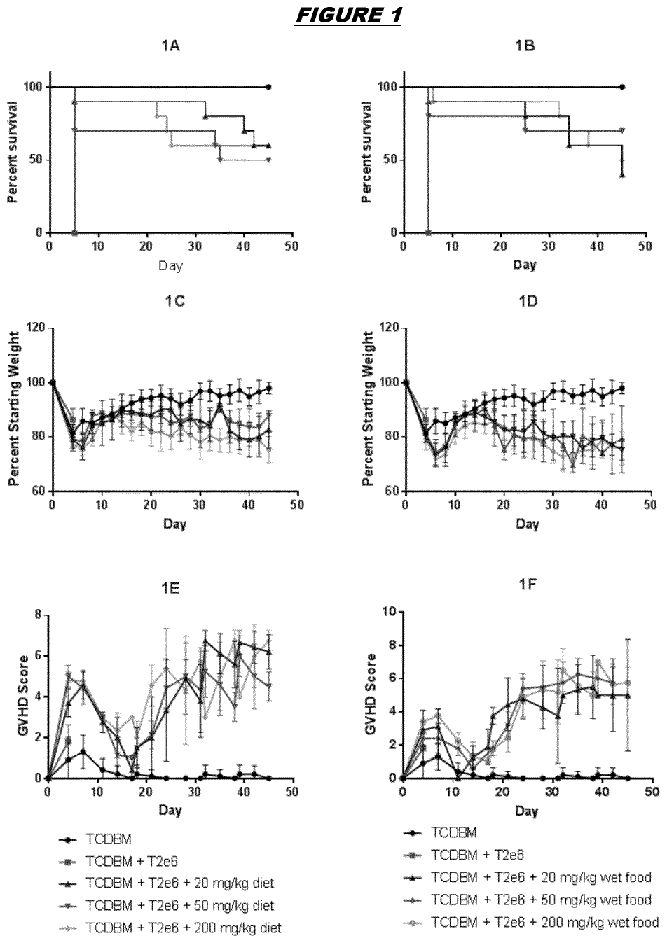

These experiments confirm that adding 2×10⁶ T cells to TCDBM (TCDBM+T2e6) results in fatal GVHD at 5 days compared to 100% survival in mice receiving TCDBM alone and show that addition of alvelestat either in pre-mixed diet pellets (FIG. 1A) or as a powder mixed with wet food (FIG. 1B) led to significant improvements in survival compared to TCDBM+T2e6 (log-rank p=0.001 TCDBM+T2e6 vs. TCDBM+T2e6+20 mg/kg diet; log-rank p=0.01 TCDBM+T2e6 vs. TCDBM+T2e6+20 mg/kg wet food). Experiments (n=5 per group) were performed in duplicate, with a total of 20 mice (n=10 diet, n=10 wet food) evaluated at each dose. It is difficult to estimate the impact on body weight or GVHD score as all control animals were deceased by day 5, though there does not appear to be a dose-response in the improvement in survival or body weight or GVHD score among the three doses tested (FIG. 1C-1F).

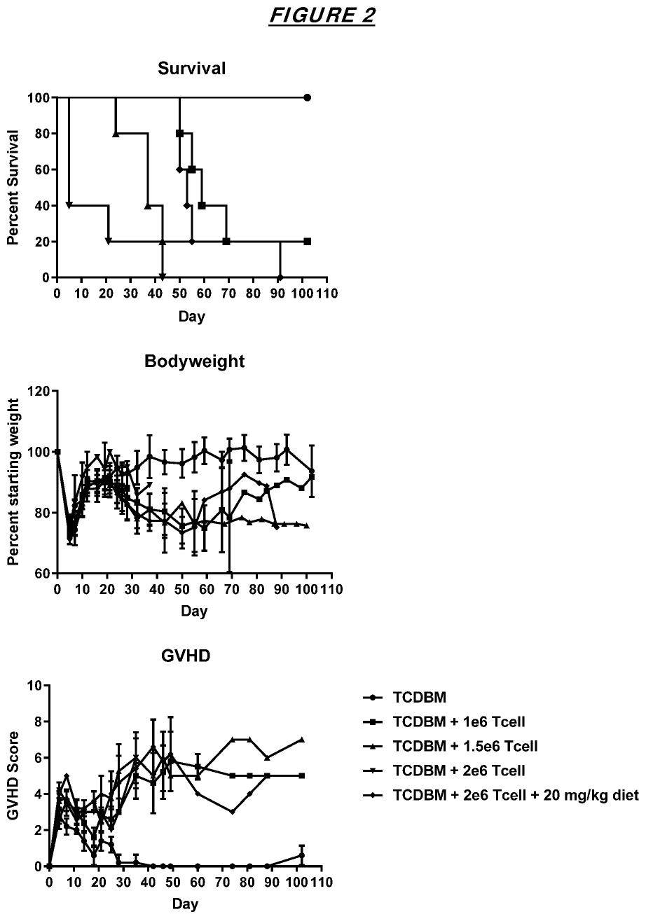

Mortality in positive control mice receiving 2×10⁶ T cells was more rapid than reported in some models (e.g. C57BL/6->BALB/c) ([4], [7], [8]), though similar to that reported with other models (e.g. C3H/Hej/C3Heb/Fej->(C3FeB6)F1) [9]. Nonetheless, to confirm that the mortality seen in FIG. 1 was driven by a T-cell mediated process, another experiment was conducted comparing mice receiving 2×10⁶ T cells to lower doses (1×10⁶ T cells and 1.5×10⁶ T cells) (FIG. 2A-C). There is a clear response to the T-cell dose, with no mortality at day 45 with 1×10⁶ T cells and more rapid mortality with higher T-cell doses (linear trend test by Cox proportional hazard model p<0.0001) (mice were followed for a longer period of time than in FIG. 1 due to the desire to observe mice receiving decreased T cells). Again, the administration of alvelestat 20 mg/kg in pre-mixed diet pellets to mice receiving 2×10⁶ T cells again significantly improved survival compared to 2×10⁶ T cells alone, with comparable results to 1×10⁶ T cells and 1.5×10⁶ T cells; this experiment is the fifth replicate in which alvelestat 20 mg/kg (either pre-mixed diet or added to wet food) resulted in a survival advantage. Because a significant survival advantage was already observed with the most severe case (2×10⁶ T cells, FIG. 1A-1B and FIG. 2A) repeated across 5 experiments, additional experiments were not performed with alvelestat and lower doses of T cells.

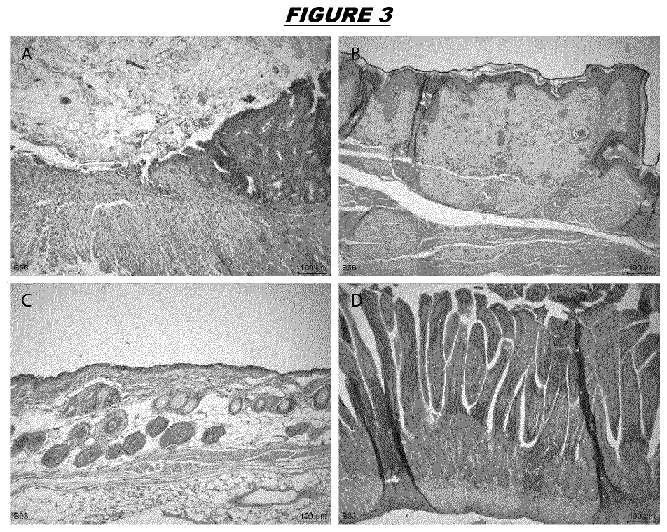

Necropsies were performed on mice after death or after they were sacrificed at the conclusion of the experiment and support the finding that that mice receiving TCDBM+T2e6 did develop GVHD (FIG. 3A-B) while those that received TCDBM alone did not have signs of GVHD (FIG. 3C-D).

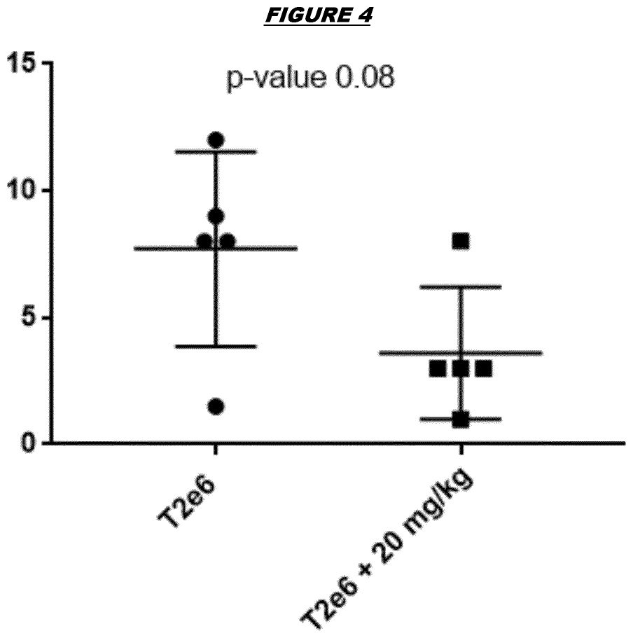

To better characterize the effects of T cells and alvelestat on organ toxicity, the above experiment was repeated with mice receiving either T2e6 or T2e6+20 mg/kg diet (n=5/group) and sacrificed at Day 4 for histological analysis. Tissues were reviewed by a blinded pathologist specializing in GVHD and scored based on architecture (crypt regeneration, surface erosion, ulceration, lamina propria inflammation, atrophy, crypt branching, endocrine cell excess, and Paneth cell excess) and epithelial cytology (vacuolization, attenuation, apoptosis, sloughing into lumen, lymphocytic infiltration, neutrophilic infiltration). Each feature was graded on a scale of 0-4 (0, normal; 0.5, focal and rare; 1, focal and mild; 2, diffuse and mild; 3, diffuse and moderate; 4, diffuse and severe) and results were summed for each organ. While no toxicity was observed in the skin or liver at Day 4 in mice receiving alvelestat, abnormalities were seen in the gut (FIG. 4), particularly crypt regeneration, columnar attenuation, and apoptosis, and there was a trend toward increased pathology in the T2e6 group (p=0.08).

In summary, these results demonstrate a significant difference in survival with alvelestat in a murine model of GVHD, supporting the use of alvelestat in preventing GVHD.

### Discussion

The above results indicate that alvelestat is effective in the treatment and prevention of GVHD. This result is highly unexpected because the main, if not exclusive, inducers of GVHD are understood to be T- and B-lymphocytes, rather than neutrophils. Extensive research has established that mature CD4⁺ and/or CD8⁺ T-cells initiate GVHD and that GVHD is dependent on a recipient's antigen presenting cells initiating alloimmune T-cell responses against the foreign histocompatibility antigens. In particular, it has been demonstrated that the depletion of αβ T cells from donor-cell inoculum prevents GVHD in rodents, humans and dogs [10].

In view of the importance of T-lymphocytes in the pathogenic mechanism of tissue damage observed during GVHD, it is surprising that alvelestat, a drug specifically targeting neutrophils, has the effect observed above against GVHD, i.e. to increase survival in GVHD models and to reduce gastrointestinal tract GVHD pathology. Neutrophils have previously been implicated in GVHD pathology but it was not known before these experiments that inhibiting neutrophil elastase would be a viable therapeutic strategy.

Based on these results, the inventors realised that alvelestat (and neutrophil elastase inhibitors more generally) will be effective in treating or preventing conditions associated with a common mechanism and pathology to GVHD.

Like GVHD, organ rejection is also principally mediated through the allorecognition of donor MHC-derived peptides by recipient CD4⁺ and CD8⁺ T-cells, with the recipient recognising the donor organ tissue antigens. The foreign antigens are presented to the recipient's immune system though donor antigen presenting cells (APCs) released from the organ that migrate to the recipient's draining lymph nodes where recipient dendritic cells process and present the alloantigens and prime T cells for activation and migration back to the organ where damage ensues. Alternatively recipient APCs pick up donor antigens and self-present [11]. In both GVHD and chronic organ rejection alloreactive T-cells are primed and generated and drive the pathogenic process. The role of the allogenic T-cells is confirmed as the common pathway by the fact that both GVHD and graft rejection can be transferred through T-cells in adoptive transfer experiments in animal models. This is further demonstrated by the clinical observations that the pathology of BOS observed due to chronic lung rejection is similar to BOS observed due to GVHD in bone marrow and stem cell transplant [12].

It has previously been reported that increased levels of elastase-derived peptides were detected in bronchoalveolar lavage fluid from LT-BOS patients [14]. However, elastase was not proposed as playing a causative role in LT-BOS, and it has not previously been thought that neutrophil elastase inhibition would provide a therapeutic strategy for treatment or prevention of organ rejection including LT-BOS. A therapeutic effect of inhibiting NE would mirror the observation as long ago as 1999 that neutrophils could play a role in LT-BOS [15].

In view of the significant results observed in GVHD, the inventors rationalised that neutrophil elastase inhibitors such as alvelestat will also be effective in treating or preventing organ rejection.

Taken together, these findings support the potential for NE inhibition using alvelestat to have a beneficial effect in treating or preventing organ rejection, in particular BOS-associated with organ rejection. This is a significant step forwards as there is currently no established therapy for these conditions.

Based on the scientific rationale and data presented above, alvelestat will be investigated in clinical trials designed to evaluate safety and efficacy in the treatment and prevention of BOS in patients following a lung transplant.

### Example 3—Prophylaxis to the Development of BOS in Patients Following Lung Transplantation

Alvelestat is administered as part of a multi-centre, randomised, standard-of-care controlled study to demonstrate efficacy and safety of alvelestat in improving survival and preventing BOS when given prophylactically to lung transplant recipients in addition to standard immunosuppressive regimen.

During the study, alvelestat is administered at a dose of up to 240 mg twice daily to patients starting immediately post-lung transplantation. Therapy continues for 2 years and may be extended up to 5 years.

Inclusion criteria:
patients who have received lung transplant (either single or double),
patients able to be consented and enrolled within 30 days after receiving the lung transplants.

Exclusion criteria:
history of heart-lung transplant, lung re-transplantation or another solid organ transplant
clinically significant stenosis unresponsive to dilation and/or stenting
active lung infection
failure of anastomosis sites

Primary endpoints:
Difference in FEV1 (percentage predicted) in alvelestat versus standard of care arm, at week 12 and week 24
Difference in BOS stage/BOS-free survival in alvelestat versus standard of care arm, at week 12 and week 24

Secondary endpoints (active compared to standard of care):
Pulmonary function as measured by mean FEV1% predicted 1 and 2 years after randomisation
BOS stage in alvelestat versus standard of care arm, at 1 and 2 years
All cause-mortality and transplant-related mortality up to 2 years
Development of RAS
Symptoms
Safety and tolerability
Duration of event-free survival corresponding to the length of time between date of randomisation and either death or occurrence of serious bacterial and viral infections that start in the lungs (defined by reporting of an SAE)

Alvelestat will show effectiveness in one or more of the above primary endpoints.

### Example 4—Treatment for BOS in Patients Following Lung Transplantation

Alvelestat is administered as part of a multi-centre, randomised, standard of care-controlled study to demonstrate efficacy and safety of alvelestat in improving BOS when given to lung transplant recipients in addition to their standard immunosuppressive regimen.

During the study, alvelestat is administered at a dose of up to 240 mg twice daily to patients who have developed BOS following lung transplant.

Inclusion criteria:
Lung transplants (either single or double)
Diagnosis of BOS>Stage 1
Other causes of lung disease have been excluded

Exclusion criteria:
Restrictive Allograft Syndrome
Patients requiring changes in immunosuppressive regimen
Active lung infection

Primary endpoint:
Difference in FEV1 (percentage predicted) in alvelestat versus standard of care arm, at week 12, 24 and 48 and 106
BOS stage in alvelestat versus standard of care arm, at week 12 and week 24

Secondary endpoints (active compared to standard of care):
Pulmonary function as measured by mean FEV1% predicted 1 and 2 years after randomisation
BOS stage in alvelestat versus standard of care arm, at 2 years
All cause-mortality and transplant-related mortality up to 2 years
Development of RAS
Symptoms
Safety and tolerability
Duration of event-free survival corresponding to the length of time between date of randomisation and either death or occurrence of serious bacterial and viral infections that start in the lungs (defined by reporting of an SAE).

Alvelestat will show effectiveness in one or more of the above primary endpoints.

### Example 5—Phase 1 Study of Alvelestat in Patients with Bronchiolitis Obliterans Syndrome (BOS) After Hematopoietic Cell Transplantation (HCT)

A Phase 1 study of alvelestat in patients with BOS after HCT was conducted.

### Methods:

Patients age 1E3 years with BOS and chronic graft-versus-host disease (GVHD) after HCT were recruited to the National Cancer Institute protocol (NCT02669251). Patients had stable systemic immunosuppression and FEV1% predicted 30% on pulmonary function tests (PFTs).

This phase 1 study had 2 parts: 8-week intra-patient dose escalation period, followed by a continuation period that allowed for up to 6 months of treatment. Alvelestat was given orally starting at 60 mg twice daily (the dose previously used in patients with chronic lung disease) and increased every 2 weeks as tolerated to 120 mg twice daily, 180 mg twice daily, and finally 240 mg twice daily. Patients continued this dose until completion of the continuation phase, or occurrence of unacceptable toxicity, dose interruption>28 days, or progression of GVHD or BOS.

The primary objective was to determine the maximum tolerated dose (MTD) based on dose-limiting toxicities. Secondary objectives included determining pharmacokinetics, markers of neutrophil elastase (NE) activity, and markers of inflammation in blood and sputum. PFTs and chronic GVHD evaluations were performed at baseline, 4 weeks, 8 weeks during the dose escalation period, and at 3 months and 6 months during the continuation period.

### Results:

7 patients were enrolled (3 men and 4 women). Median FEV1 after bronchodilator at time of enrolment was 44% (range 38-74).

All 7 patients were able to tolerate dose escalation of alvelestat up to the maximum dose 240 mg twice daily; MTD was not reached. The most common adverse events (AEs) that were possibly related to study treatment were all grade 2, and included increased creatinine (3 patients), ALT or AST elevation (3 patients), and upper respiratory infection (3 patients). The only grade 3 AEs that were possibly related to study drug were gastroenteritis and vomiting requiring hospitalization in 1 patient and pneumonia in 1 patient.

Three patients completed the study with 8 weeks+6 months of treatment. Four patients required dose interruptions, and only 1 of those required dose reduction for grade 3 gastroenteritis (resulting in dehydration and elevated creatinine). Four patients discontinued treatment prior to end of study: 2 patients had dose interruption of >28 days due to adverse events, 1 patient had a decline in FEV1 after pneumonia, and treatment was stopped in 1 patient due to investigator discretion.

The median duration of treatment was 6.4 months. Based on NIH chronic GVHD consensus criteria, 6 patients had unchanged disease and 1 patient had progressive disease (decline in FEV1 after pneumonia). Although patients did not achieve the 10% improvement in FEV1 required for an organ response, 2 patients had improvement of 9% in FEV1 and 4 patients had improvement in the Lee chronic GVHD symptom scale lung score.

Preliminary pharmacokinetic analyses of the 7 patients showed a linear dose-dependent increase in each exposure metric (steady-state trough and steady-state peak), despite some inter-patient variability. Bronchoalveolar lavage fluid and induced sputum samples are being analyzed for NE activity.

### Conclusion:

In this phase 1 study of the oral NE inhibitor, alvelestat, in patients with BOS after HCT, MTD was not reached and the study drug was well tolerated. Six patients had stable disease, while 1 patient had progression in the setting of pneumonia. Two patients notably had improvement in FEV1 of 9%, and 4 patients experienced improvement in lung symptoms at some point on treatment, 2 during the 6 month treatment period assessment and 2 at the end of the study treatment period. We have demonstrated that NE inhibition is well tolerated and shows a signal of stabilizing disease in patients with advanced BOS.

The ability to improve lung function and/or to prevent progression of disease and further deterioration of lung function may lead to significant improvements in treatment of GVHD, in particular cGVHD, and related conditions.

These results further support the use of alvelestat in the methods of the invention, including relating to treatment or prevention of graft rejection, LT-BOS, and GVHD.

TABLE 1
Patient and disease characteristics.

| | | | | | | | | | LSS* score |
|---|---|---|---|---|---|---|---|---|---|
| | | | | | | End of | LSS* score | LSS* score | for lung/ |
| | | | | cGVHD | Base- | treatment | for lung/ | for lung/ | breathing at |
| Patient | | | cGVHD | involved | line | FEV1 % | breathing at | breathing at | the end of |
| # | Age | Gender | severity | organs | FEV1 | predicted | C1 | C5 | treatment |
| 1 | 46 | F | Moderate | Lungs, mouth | 74% | 73% | 3 | 3 | 1 |
| 2 | 21 | F | Severe | Lungs | 38% | 47% | 7 | 7 | 8 |
| 3 | 50 | M | Severe | Lungs, mouth, | 46% | 55% | 3 | 3 | 2 |
| | | | | esophagus, | | | | | |
| | | | | skin, eyes, | | | | | |
| | | | | joints/fascia | | | | | |
| 4 | 44 | M | Severe | Lungs, eyes | 53% | 40% | 11 | 11 | 11 |
| 5 | 62 | M | Severe | Lungs | 52% | 46% | 3 | 2 | 3 |
| 6 | 59 | F | Severe | Lungs, skin, | 44% | 38% | 4 | 5 | 7 |
| | | | | eyes, genital | | | | | |
| 7 | 61 | F | Severe | Lungs, skin | 44% | 41% | 5 | 4 | 5 |

*Lee cGVHD Symptom Scale [17]. Time point C1 is the baseline before treatment. Time point C5 is the continuation dosing period up to 6 months.

### REFERENCES

[1] Verleden S E, Vos R, Vanaudenaerde B M, Verleden G M, J Thorac Dis 2017;9(8):2650-2659

[2] Meyer K C, Raghu G, Verleden G M, Corris P A, Aurora P, Wilson K C, Brozek J, Glanville A R and the ISHLT/ATS/ERS BOS Task Force Committee; Eur Respir J 2014; 44: 1479-1503

[3] Stevens T, Ekholm K, Granse M, Lindahl M, Kozma V, Jungar C, Ottosson T, Falk-Hakansson H, Churg A, Wright J L, Lal H, Sanfridson A. AZD9668: pharmacological characterization of a novel oral inhibitor of neutrophil elastase. The Journal of pharmacology and experimental therapeutics. 2011; 339(1):313-20. Epub 2011/07/28. doi: 10.1124/jpet.111.182139. PubMed PMID: 21791628.

[4] Chen B J, Cui X, Liu C, Chao N J. Prevention of graft-versus-host disease while preserving graft-versus-leukemia effect after selective depletion of host-reactive T cells by photodynamic cell purging process. Blood. 2002; 99(9):3083-8. Epub 2002/04/20. PubMed PMID: 11964269.

[5] Delbosc S, Rouer M, Alsac J M, Louedec L, Philippe M, Meilhac O, Whatling C, Michel J B. Elastase inhibitor AZD9668 treatment prevented progression of experimental abdominal aortic aneurysms. Journal of vascular surgery. 2016; 63(2):486-92.e1. Epub 2014/09/02. doi: 10.1016/j.jvs.2014.07.102. PubMed PMID: 25175632.

[6] Zhang P, Wu J, Deoliveira D, Chao N J, Chen B J. Allospecific CD4(+) effector memory T cells do not induce graft-versus-host disease in mice. Biology of blood and marrow transplantation: journal of the American Society for Blood and Marrow Transplantation. 2012;18(10):1488-99. Epub 2012/07/20. doi: 10.1016/j.bbmt.2012.07.009. PubMed PMID: 22809867; PMCID: PMC3443280.

[7] Chen B J, Deoliveira D, Cui X, Le N T, Son J, Whitesides J F, Chao N J. Inability of memory T cells to induce graft-versus-host disease is a result of an abortive alloresponse. Blood. 2007;109(7):3115-23. Epub 2006/12/07. doi: 10.1182/blood-2006-04-016410. PubMed PMID: 17148592; PMCID: PMC1852216.

[8] Chen B J, Cui X, Sempowski G D, Liu C, Chao N J. Transfer of allogeneic CD62L-memory T cells without graft-versus-host disease. Blood. 2004;103(4):1534-41. Epub 2003/10/11. doi: 10.1182/blood-2003-08-2987. PubMed PMID: 14551132.

[9] Cooke K R, Hill G R, Crawford J M, Bungard D, Brinson Y S, Delmonte J, Jr., Ferrara J L. Tumor necrosis factor-alpha production to lipopolysaccharide stimulation by donor cells predicts the severity of experimental acute graft-versus-host disease. The Journal of clinical investigation. 1998;102(10):1882-91. Epub 1998/11/20. doi: 10.1172/jci4285. PubMed PMID: 9819375; PMCID: PMC509139.

[10] Schlomchik W D, Nature Reviews Immunology, vol. 7, pages 340-352 (2007).

[11] Nath D S, Basha H I, Mohanakumar T, Curr. Opin. Organ Transplant. 2010 February; 15(1): 16-20; doi:10.1097/MOT.0b013e3283342780

[12] Verleden S E, Mcdonough J, Schoemans H, Knoop C, Verschakelen J, Dubbledam A, Boone M, Van Hoorebeke L, Verbeken E, Weynand B, Van Raemdonck D, Verleden G, Vos R, Vanaudenaerde B, The Journal of Heart and Lung Transplantation, 38(4), S407-S408

[13] Groutas W C, Dou D, Alliston K R, Expert Opin Ther Pat. 2011 March ; 21(3):

339-354

[14] Stone M D, Harvey S B, Nelsestuen G L, Reilly C, Hertz M I, Wendt C H; PLOS ONE, 2 Jan. 2014, Volume 9, Issue 1, e84471, pages 1-6

[15] Hirsch J et al, Am J Respir Crit Care Med Vol 160. pp 1640-1646,1999.

[16] Lee S J, Blood (2017) 129 (1): 30-37.

[17] Lee S J et al; Biol Blood Marrow Transplant 2002;8(8):444-52

1. A method for treating or preventing graft rejection, comprising administering an effective amount of alvelestat or a pharmaceutically acceptable salt and/or solvate thereof to a subject in need thereof.

2. The method of claim 1 wherein the graft comprises one or more organs selected from the group consisting of a kidney, heart, liver, lung and pancreas.

3. The method of claim 1 wherein the graft comprises a lung.

4. The method of any preceding claim wherein the subject has, or is at risk of having, lung transplant associated bronchiolitis obliterans syndrome.

5. The method of any preceding claim wherein the graft rejection is chronic graft rejection.

6. The method of any of claims 1-4 wherein the graft rejection is acute graft rejection.

7. A method for treating or preventing lung transplant associated bronchiolitis obliterans syndrome, comprising administering an effective amount of alvelestat or a pharmaceutically acceptable salt and/or solvate thereof to a subject in need thereof.

8. A method for treating or preventing graft versus host disease (GVHD), comprising administering an effective amount of alvelestat or a pharmaceutically acceptable salt and/or solvate thereof in a subject in need thereof.

9. The method of claim 8 wherein the GVHD is chronic GVHD (cGVHD).

10. The method of claim 8 wherein the GVHD is acute GVHD (aGVHD).

11. The method of any of claims 8 to 10 wherein the GVHD manifests after bone marrow transplantation.

12. The method of any one of claims 8 to 11 wherein the GVHD manifests after hematopoietic stem cell transplantation.

13. The method of any one of claims 8 to 12 wherein the GVHD is characterised by damage to one or more selected from the group consisting of the eyes, joints, fascia, genital organ, lung, liver, skin, or gastrointestinal tract (e.g. mouth, oesophagus).

14. The method of any one of claims 8 to 12 wherein the GVHD is characterised by damage to one or more selected from the group consisting of the lung, liver, skin, or gastrointestinal tract.

15. The method of any one of claims 8 to 14 where in the subject has moderate or severe cGVHD.

16. The method of any one of claims 8 to 15 wherein the subject has, or is at risk of having, bronchiolitis obliterans syndrome.

17. A method for treating or preventing bronchiolitis obliterans syndrome (BOS) associated with GVHD, comprising administering an effective amount of alvelestat or a pharmaceutically acceptable salt and/or solvate thereof to a subject in need thereof.

18. The method of claim 17 wherein the BOS is associated with hematopoietic stem cell transplant.

19. The method of claim 17 wherein the BOS is associated with bone marrow transplant.

20. The method of any preceding claim wherein alvelestat or a pharmaceutically acceptable salt and/or solvate thereof is administered prior to transplantation into the subject.

21. The method of any one of claims 1 to 1516 wherein alvelestat or a pharmaceutically acceptable salt and/or solvate thereof is administered after transplantation into the subject.

22. The method of any preceding claim wherein the treatment or prevention comprises inhibiting neutrophil elastase.

23. The method of any preceding claim wherein the treatment or prevention comprises improving or preventing worsening of the FEV1% predicted in the subject.

24. The method of any preceding claim wherein the treatment or prevention comprises improving or preventing worsening of the BOS grade of the subject.

25. The method of any preceding claim wherein the treatment of cGVHD comprises improving the cGVHD severity score in a subject.

26. The method of any preceding claim wherein the treatment of cGVHD comprises improving the Lee cGVHD Symptom Scale in a subject, in particular the Lee cGVHD Symptom Scale lung score in a subject with cGVHD affecting a lung.

27. The method of any preceding claim comprising improving lung function in a subject.

28. The method of any preceding claim comprising preventing worsening of lung function in a subject.

29. The method of any preceding claim comprising preventing progression or worsening of disease in a subject.

30. The method of any preceding claim wherein alvelestat is in the form of the free base.

31. The method of any preceding claim wherein alvelestat is in the form of alvelestat tosylate.

32. The method of any preceding claim comprising administering alvelestat or a pharmaceutically acceptable salt and/or solvate thereof twice daily.

33. The method of any preceding claim comprising administering alvelestat or a pharmaceutically acceptable salt and/or solvate thereof at a dose of alvelestat of up to 240 mg twice daily.

34. The method of any preceding claim comprising administering alvelestat or a pharmaceutically acceptable salt and/or solvate thereof at a dose of alvelestat of 60 mg, 120 mg, 180 mg or 240 mg twice daily.

35. The method of any preceding claim comprising administering alvelestat or a pharmaceutically acceptable salt and/or solvate thereof at a dose of alvelestat of 240 mg twice daily.

36. The method of any preceding claim comprising administering alvelestat or a pharmaceutically acceptable salt and/or solvate thereof at a dose of alvelestat of 60 mg twice daily for a first period of time, followed by 120 mg twice daily for a second period of time, followed by 180 mg twice daily for a third period of time, and 240 mg twice daily thereafter.

37. The method of any preceding claim comprising administering alvelestat or a pharmaceutically acceptable salt and/or solvate thereof at a dose of alvelestat of 60 mg twice daily for two weeks, followed by 120 mg twice daily for two weeks, followed by 180 mg twice daily for two weeks, and 240 mg twice daily thereafter.

38. The method of any preceding claim comprising administering alvelestat or a pharmaceutically acceptable salt and/or solvate thereof by oral administration.

39. The method of any preceding claim, further comprising administering to the subject one or more immunosuppressive agents.
